(19) United States
(12) Patent Application Publication
(10) Pub. No.: US 2008/0281091 A1
(43) Pub. Date: Nov. 13, 2008

(54) Process for the Preparation of Porphyrin Derivatives as Antimicrobial Agents by Photodynamic Therapy (Pdt)

(76) Inventors: Derek Brundish, West Sussex (GB); William Love, West Sussex (GB); William Rhys-Williams, West Susex (GB); Xiang Dong Feng, Qingdao (CN); Frederic Naud, Binningen (CH); Hans Meier, Basel (CH)

Correspondence Address: PATREA L. PABST;PABST PATENT GROUP LLP, 400 COLONY SQUARE, SUITE 1200, 1201 PEACHTREE STREET, ATLANTA, GA, 30361, US

(21) Appl. No.: 12/158,779

(22) PCT Filed: Dec. 22, 2006
(86) PCT No.: PCT/GB06/04920

§ 371 Date: Jun. 23, 2008

(30) Foreign Application Priority Data
Dec. 24, 2005 (GB) 0526474.2

(51) Int. Cl. C07D 487/22

(52) U.S. Cl. 540/145

(57) ABSTRACT

There is provided a process for the preparation of 5,15-bis-[4-(3-trimethylammonio-propyloxy)-phenyl]-porphyrin dihalide, wherein the process comprises step (a) of providing 4-(3-bromopropyloxy)benzaldehyde, step (b) of providing dipyrrolmethane, step (c) of reacting the 4-(3-bromopropyloxy)benzaldehyde with the dipyrrol-methane, together with trifluoro acetic acid, in the presence of an oxidation reagent to produce 5,15-bis-[4-(3-bromo-propyloxy)-phenyl]-porphyrin which is purified by Soxhlet extraction from the adsorbed state on a bed of alumina under highly controlled conditions; and step (d) of reacting the 5,15-bis-[4-(3-bromo-propyloxy)-phenyl]-porphyrin with trimethylamine in the presence of dry dimethylformamide to produce 5,15-bis-[4-(3-trimethylammonio-propyl-oxy)-phenyl]-porphyrin dibromide. In a preferred embodiment, the process further comprises step (e) of passing the 5,15-bis-[4-(3-trimethylammonio-propyloxy)-phenyl]-porphyrin dibromide produced in step (d) through an anion exchanger to produce 5,15-bis-[4-(3-trimethylammonio-propyloxy)-phenyl]-porphyrin dichloride. There is provided a process for the preparation of 5,15-bis-(4-{3-[(3-dimethylamino-propyl)-dimethyl-ammonio]-propyloxy}-phenyl]-porphyrin dihalide.

### CROSS-REFERENCE TO RELATED APPLICATIONS

This application is a National Stage of International Application No. PCT/GB2006/004920 filed on Dec. 22, 2006, which claims priority to GB Application No. 0526474.2 filed on Dec. 24, 2005, all of which are herein incorporated in their entirety by reference.

### FIELD

The invention relates to a novel process for the preparation of halide salts of 5,15-bis-[4-(3-trimethylammonio-propyloxy)-phenyl]-porphyrin and 5,15-bis-(4-{3-[(3-dimethylamino-propyl)-dimethyl-ammonio]-propyloxy}-phenyl]-porphyrin, and in particular the dichloride salts thereof.

### BACKGROUND

The resistance to antibiotics developed by an increasing number of microorganisms is recognised to be a worldwide health problem (Tunger et al., 2000, Int. J. Microb. Agents 15:131-135; Jorgensen et al., 2000, Clin. Infect. Dis. 30:799-808). Thus, the development of non-antibiotic approaches for killing microorganisms is urgently required for controlling antibiotic-untreatable infections and limiting the development of additional antibiotic-resistant strains.

The treatment of microbial infections by photodynamic therapy (PDT) represents a valuable alternative method for eradicating bacteria since it involves a mechanism which is markedly different from that typical of most antibiotics. PDT is based on the use of a photosensitising molecule that, once activated by light, generates oxygen reactive species that are toxic for a large variety of prokaryotic and eukaryotic cells including bacteria, mycoplasmas and yeasts (Malik et al., 1990, J. Photochem. Photobiol. B Biol. 5:281-293; Bertoloni et al., 1992, Microbios 71:33-46). Importantly, the photosensitising activity of many photodynamic agents against bacteria is not impaired by the resistance to antibiotics but, instead, depends mainly on their chemical structure (Malik et al., 1992, J. Photochem. Photobiol B Biol. 14:262-266).

Various types of neutral and anionic photosensitising agents exhibit a pronounced phototoxic activity against Gram-positive bacteria. However, such photosensitising agents exert no appreciable cytotoxic activity against Gram-negative bacteria unless the permeability of the outer membrane is altered by treatment with ethylene diamine tetra-acetic acid (EDTA) or polycations (Bertoloni et alt, 1990, FEMS Microbiol Lett. 71: 149-156; Nitzan et al., 1992, Photochem. Photobiol. 55:89-97). It is believed that the cellular envelope of Gram negative bacteria, which is more complex and thicker than that of Gram positive bacteria, prevents an efficient binding of the photosensitising agent or intercepts and deactivates the cytotoxic reactive species photogenerated by the photosensitising agent (Ehrenberg et al., 1985, Photochem. Photobiol. 41:429-435; Valduga et al., 1993, J. Photochem. Photobiol. B. Biol. 21:81-86).

In contrast, positively charged (cationic) photosensitising agents, including porphyrins and phthalocyanines, promote efficient inactivation of Gram-negative bacteria without the need for modifying the natural structure of the cellular envelope (Merchat et al., 1996, J. Photochem. Photobiol. B. Biol. 32:153-157; Minnock et al., 1996, J. Photochem. Photobiol. B. Biol. 32:159-164). It appears that the positive charge favours the binding of the photosensitising agent at critical cellular sites that, once damaged by exposure to light, cause the loss of cell viability (Merchat et al., 1996, J. Photochem. Photobiol. B. Biol. 35:149-157). Thus, it has been reported that Escherichia coli is efficiently inactivated by visible light after incubation with the cationic 5,10,15,20-tetrakis-(4-N-methylpyridyl)porphine (T₄ MPyP) (Valduga et al., 1999, Biochem. Biophys. Res. Commun. 256:84-88). The phototoxic activity of this porphyrin is mainly mediated by the impairment of the enzymic and transport functions of both the outer and cytoplasmic membranes, rather than by binding to DNA.

However, the utility of known porphyrin-based photodynamic therapy agents is limited due to their toxicity against mammalian host tissue cells, i.e. the compounds are unable to differentiate between target microbial cells and host cells. In addition, the utility of known porphyrin-based photodynamic therapy agents is further limited by their relatively low potency for target microbial cells.

Porphyrin-based compounds with improved toxicity profiles and high potency, which can be used in PDT to kill microbial cells preferentially, are described in WO 2004/056828. A particularly preferred compound described therein is 5,15-bis-[4-(3-trimethylammonio-propyloxy)-phenyl]-porphyrin. However, the syntheses disclosed in WO 2004/056828 are small-scale, suitable only for research purposes.

Further porphyrin-based compounds for use in PDT are disclosed in WO 2004/035590. However, the syntheses disclosed therein are again only small-scale and, additionally, the product yields are low.

Hence, there exists a need for improved synthesis routes for porphyrin-based compounds for use in PDT which allow commercially useful quantities of the compounds to be produced.

In the preparation of such drug substances, it is desirable to minimise the cost of producing the substance whilst, at the same time, utilising a preparative route that meets modern environmental and health and safety standards.

Modifications to a preparative route that could result in a decreased overall cost include:
(a) improvements in the yield(s) of one or more steps;
(b) a reduction in the number of synthetic steps and/or unit operations used;
(c) a decrease in the quantities of reagents and/or solvents employed;
(d) specific measures to accommodate findings which are novel in the field of study;
(e) minimisation of the amount of energy expended (e.g. through elimination or reduction of the need for heating or cooling); and/or
(f) a shortening of the total time required to complete the preparative route.

The present invention seeks to provide a method, suitable for large-scale production in high yield, for the preparation of halide salts of 5,15-bis-[4-(3-trimethylammonio-propyloxy)-phenyl]-porphyrin.

The present invention further seeks to address the problem of contamination of the desired product with the 10,20-dichloro analogue of the desired product, which forms as the product of a side reaction.

### SUMMARY OF THE INVENTION

According to a first aspect of the invention, there is provided a process for the preparation of 5,15-bis-[4-(3-trimethylammonio-propyloxy)-phenyl]-porphyrin dihalide, wherein the process comprises the following steps:
(a) providing 4-(3-bromopropyloxy)benzaldehyde;
(b) providing dipyrrolmethane;
(c) reacting the 4-(3-bromopropyloxy)benzaldehyde with the dipyrrolmethane, together with trifluoroacetic acid, in the presence of an oxidation reagent to produce 5,15-bis-[4-(3-bromo-propyloxy)-phenyl]-porphyrin; and
(d) reacting the 5,15-bis-[4-(3-bromo-propyloxy)-phenyl]-porphyrin with trimethylamine in the presence of dry dimethylformamide to produce 5,15-bis-[4-(3-trimethylammonio-propyloxy)-phenyl]-porphyrin dibromide
   wherein in step (c) the 5,15-bis-[4-(3-bromo-propyloxy)-phenyl]-porphyrin is purified by Soxhlet extraction.

A preferred embodiment of the first aspect of the invention provides a process for the preparation of 5,15-bis-[4-(3-trimethylammonio-propyloxy)-phenyl]-porphyrin dihalide, wherein the process comprises the following steps:
(a) providing 4-(3-bromopropyloxy)benzaldehyde;
(b) providing dipyrrolmethane;
(c) reacting the 4-(3-bromopropyloxy)benzaldehyde with the dipyrrol-methane, together with trifluoroacetic acid;
(d) adding an oxidation reagent to produce 5,15-bis-[4-(3-bromo-propyloxy)phenyl]-porphyrin;
(e) purifying the 5,15-bis-[4-(3-bromo-propyloxy)-phenyl]-porphyrin produced in step (d) by Soxhlet extraction in the presence of aluminium oxide; and
(f) reacting the purified 5,15-bis-[4-(3-bromo-propyloxy)-phenyl]-porphyrin with trimethylamine in the presence of dry dimethylformamide to produce 5,15-bis-[4-(3-trimethylammonio-propyloxy)-phenyl]-porphyrin dibromide
   wherein step (e) comprises monitoring of Soxhlet extracted fractions to determine the presence therein of contaminants.

In a further preferred embodiment, the process further comprises step (g) of passing the 5,15-bis-[4-(3-trimethylammonio-propyloxy)-phenyl]-porphyrin dibromide produced in step (d) through an anion exchanger to produce 5,15-bis-[4-(3-trimethylammonio-propyloxy)-phenyl]-porphyrin dichloride.

Preferred features of the reaction steps of the process of the invention are described below.

### Step (a)

Step (a) comprises the provision of 4-(3-bromopropyloxy)benzaldehyde.

It will be appreciated by persons skilled in the art that the 4-(3-bromopropyloxy)benzaldehyde should be as pure as possible. Preferably, the 4-(3-bromopropyloxy)benzaldehyde has a purity of at least 85%, for example at least 90%, 95%, 96%, 97%, 98%, 99% or 100% pure. For example, the 4-(3-bromopropyloxy)benzaldehyde may have a purity of at least 95, preferably between 95 and 98%.

In a preferred embodiment of the method of the invention, step (a) comprises preparation of the 4-(3-bromopropyloxy)-benzaldehyde by reaction of 4-hydroxybenzaldehyde and 1,3-dibromopropane in an inert atmosphere (for example, under argon).

Advantageously, the 4-hydroxybenzaldehyde and 1,3-dibromopropane are reacted in a molar ratio of between 1:4 and 1:6, preferably in a molar ratio of 1:5.

Suitable solvents for performing the reaction will be known to those skilled in the art. Conveniently, the reaction is performed using anhydrous acetonitrile as a solvent.

The reaction is preferably carried out at a temperature of 20° C. or above (e.g. 25, 30, 35, 40, 45 or, particularly, 50° C. or above), such as any temperature from 40 to 70° C., e.g. from 45, 50 or 55 to 65° C., or, particularly, from 50 to 60° C. Most preferably, the reaction is performed at a temperature of between 55 and 60° C. Conveniently, the reaction is performed for between 3 to 4 hours.

As soon as the 4-hydroxybenzaldehyde has been consumed the reaction may be cooled to room temperature. The progression of the reaction may conveniently be monitored by gas chromatography.

Upon completion of the reaction, the 4-(3-bromopropyloxy)benzaldehyde may be purified from the reaction mixture by methods well known in the art. For example, the product may be purified by removal of solids by filtration, reduction of the solvent volume by rotary evaporation and removal of excess 1,3-dibromopropane by high vacuum distillation.

Preferably, the 4-(3-bromopropyloxy)benzaldehyde is further purified by column chromatography under argon and pooling of elution fractions containing the product.

The percentage yield of 4-(3-bromopropyloxy)benzaldehyde in the reaction described above is preferably greater than 50%, for example greater than 55%, greater than 60%, greater than 65%, greater than 70%, greater than 75%, greater than 80%, greater than 85%, greater than 90% or greater than 95%. Advantageously, the yield is at least 75%.

Likewise, the mass of 4-(3-bromopropyloxy)benzaldehyde produced in the reaction described above is preferably greater than 100 g, for example greater than 200 g, greater than 300 g, greater than 400 g, greater than 500 g, greater than 600 g, greater than 700 g, greater than 800 g, greater than 900 g, or greater than 1 kg. Advantageously, the mass of product is at least 900 g.

### Step b)

Step (b) comprises the provision of dipyrrolmethane. For example, dipyrrolmethane may be produced using the method of Laha et al. (2003) Org. Proc. Res. Devel. 7:799-812.

As in the case of Step (a) above, it will be appreciated by persons skilled in the art that the dipyrrolmethane should be as pure as possible. Preferably, the dipyrrolmethane has a purity of at least 85%, for example at least 90%, 95%, 96%, 97%, 98%, 99% or 100% pure. More preferably, the dipyrrolmethane has a purity of at least 85%, for example between 85 and 99%.

In a preferred embodiment of the method of the invention, step (b) comprises preparation of dipyrrolmethane by reaction of pyrrole with paraformaldehyde in an inert atmosphere (for example, under argon).

Advantageously, the pyrrole and paraformaldehyde are reacted in a molar ratio of between 120:1 and 80:1, preferably in a molar ratio of 100:1.

Suitable catalysts for the reaction of pyrrole with paraformaldehyde include indium-based catalysts and trifluoroacetic acid. Preferably, the reaction is catalysed by indium trichloride.

The reaction is preferably carried out at a temperature of 20° C. or above (e.g. 25, 30, 35, 40, 45 or, particularly, 50° C. or above), such as any temperature from 40 to 70° C., e.g. from 45, 50 or 55 to 65° C., or, particularly, from 50 to 60° C. Most preferably, the reaction is performed at a temperature of between 50 and 55° C.

The progression of the reaction may conveniently be monitored by gas chromatography. Upon completion of the reaction, the reaction mixture is cooled to room temperature before addition of sodium hydroxide.

The dipyrrolmethane may be purified from the reaction mixture by methods well known in the art. For example, the product may be purified by removal of solids by filtration, removal of excess pyrrole from the filtrate by rotary evaporation and then drying under high vacuum.

Optionally, the dipyrrolmethane is purified by column chromatography and pooling of elution fractions containing the product. Alternatively, the dipyrrolmethane may be purified by solid distillation. The dipyrrolmethane may be further purified by recrystallisation.

The percentage yield of dipyrrolmethane in the reaction described above is preferably greater than 50%, for example greater than 55%, greater than 60%, greater than 65%, greater than 70%, greater than 75%, greater than 80%, greater than 85%, greater than 90% or greater than 95%. Advantageously, the yield is at least 80%.

Likewise, the mass of dipyrrolmethane produced in the reaction described above is preferably greater than 10 g, for example greater than 20 g, greater than 30 g, greater than 40 g, greater than 50 g, greater than 60 g, greater than 70 g, greater than 80 g, greater than 90 g, or greater than 100 g. Advantageously, the mass of product is at least 60 g.

### Steps (c) to (e)

Steps (c) to (e) comprise reacting the 4-(3-bromopropyloxy)benzaldehyde with the dipyrrolmethane, together with trifluoroacetic acid, in the presence of an oxidation reagent to produce 5,15-bis-[4-(3-bromo-propyloxy)-phenyl]-porphyrin

It will be appreciated by persons skilled in the art that the reaction of steps (c) to (e) should be performed in the dark and in the absence of oxygen (for example, under argon).

Suitable solvents for use in steps (c) to (e), such as dichloromethane, are well known in the art.

In a preferred embodiment of the process of the invention, the 4-(3-bromopropyloxy)benzaldehyde and dipyrrolmethane are reacted in a molar ratio of 1:1.

Preferably, the 4-(3-bromopropyloxy)benzaldehyde and dipyrrolmethane are reacted at a concentration of between 7 and 10 mmol/L of both reagents, for example 8.75 mmol/L.

It will be appreciated that the oxidation reagent should be added after the macrocycle has been formed. Advantageously, the oxidation reagent in step (d) is added after the reaction mixture has been stirred at room temperature for at least 12 hours, preferably for at least 16 hours.

Suitable oxidation reagents are well known in the art, for example air, O₂/Pt, H₂O₂, p-chloranil and 2,3-dichloro-5,6-dicyano-1,4-benzoquinone (DDQ). Preferably, however, the oxidation reagent is DDQ.

Upon completion of the oxidation reaction, the reaction mixture may be neutralised, for example by the addition of triethylamine. Preferably, neutralisation occurs within 1 hour of addition of the oxidation reagent.

Alumina (aluminium oxide) may also be added to the reaction mixture, preferably within 20 minutes of neutralisation.

Following addition of neutral alumina, the reaction mixture is then dried, for example by rotary evaporation. Preferably, the rotary evaporation is performed at a temperature not exceeding about 40° C.

The 5,15-bis-[4-(3-bromo-propyloxy)-phenyl]-porphyrin product is then recovered from the adsorbed state by Soxhlet extraction under highly-defined conditions making use of an essential in-process control analysis.

Conveniently, the Soxhlet extraction is performed with dichloromethane at 80° C., preferably for 5 to 6 days. Alternatively, the product may be purified by filtration through alumina (but this is typically less efficient and does not allow the preferential removal of the chlorinated side-products that may then continue to accumulate).

In one embodiment, the in-process monitoring in step (e) is performed by HPLC. Advantageously, the in-process monitoring comprises assaying for the presence of the 10,20-dichloro analogue of 5,15-bis-[4-(3-bromo-propyloxy)-phenyl]-porphyrin.

Preferably, Soxhlet extracted fractions comprising more than 0.5% of the 10,20-dichloro analogue of 5,15-bis-[4-(3-bromo-propyloxy)-phenyl]-porphyrin are discarded prior to step (f).

Upon completion of the Soxhlet extraction, the volume of solvent (dichloromethane) is reduced by rotary evaporation. The 5,15-bis-[4-(3-bromo-propyloxy)-phenyl]-porphyrin may then be crystallised and collected by filtration.

The percentage yield of 5,15-bis-[4-(3-bromo-propyloxy)-phenyl]-porphyrin in the reaction described above is preferably greater than 20%, for example greater than 25%, greater than 30%, greater than 35%, greater than 40%, greater than 45%, greater than 50%, greater than 55%, greater than 60% or greater than 70%. Advantageously, the yield is at least 45%.

Likewise, the mass of 5,15-bis-[4-(3-bromo-propyloxy)-phenyl]-porphyrin produced in the reaction described above is preferably greater than 10 g, for example greater than 20 g, greater than 30 g, greater than 40 g, greater than 50 g, greater than 60 g, greater than 70 g, greater than 80 g, greater than 90 g, or greater than 100 g. Advantageously, the mass of product is at least 35 g.

A specification is set for 5,15-bis-[4-(3-bromo-propyloxy)-phenyl]-porphyrin.

### Step (f)

Step (f) comprises reacting 5,15-bis-[4-(3-bromo-propyloxy)-phenyl]-porphyrin with trimethylamine in the presence of dry dimethylformamide to produce 5,15-bis-[4-(3-trimethylammonio-propyloxy)-phenyl]-porphyrin dibromide.

Conveniently, the dimethylformamide has been pre-treated with a molecular sieve in order to ensure optimal dryness.

Advantageously, the 5,15-bis-[4-(3-bromo-propyloxy)-phenyl]-porphyrin and trimethylamine are reacted in a molar ratio of 1:150 to 1:250, for example in a molar ratio of 1:200.

Preferably, the 5,15-bis-[4-(3-bromo-propyloxy)-phenyl]-porphyrin is reacted at a concentration of between 3 mmol/L and 5 mmol/L, for example 4 mmol/L.

As in steps (a) to (e), it is important to perform the reaction of step (f) in an inert atmosphere, for example under argon.

Preferably, the reaction vessel is heated and, optionally, under pressure. For example, the reaction may be performed at a temperature of 40° C. or above (in particular, 50° C.) and a pressure of 1 to 2 bar. Conveniently, the reaction is allowed to proceed for at least 10 hours, for example at least 12, 14, 16, 18 or 20 hours.

In a preferred embodiment, the reaction in step (f) is performed in an autoclave. However, care should be taken in selection of the autoclave as the reaction product is a co-ordinator for many metal ions. Most preferably, the autoclave chamber is constructed of glass, although Hastelloy C and E metals are also suitable.

Upon completion of the reaction (which may be monitored by LC/MS), the reaction mixture is cooled. The excess trimethylamine may then be removed, for example under vacuum.

The reaction product, 5,15-bis-[4-(3-trimethylammonio-propyloxy)-phenyl]-porphyrin dibromide, may then be collected by filtration.

The percentage yield of 5,15-bis-[4-(3-trimethylammonio-propyloxy)-phenyl]-porphyrin dibromide in the reaction described above is preferably greater than 50%, for example greater than 55%, greater than 60%, greater than 65%, greater than 70%, greater than 75%, greater than 80%, greater than 85%, greater than 90% or greater than 95%, Advantageously, the yield is at least 95%.

Likewise, the mass of 5,15-bis-[4-(3-trimethylammonio-propyloxy)-phenyl]-porphyrin dibromide produced in the reaction described above is preferably greater than 10 g, for example greater than 20 g, greater than 30 g, greater than 40 g, greater than 50 g, greater than 60 g, greater than 70 g, greater than 80 g, greater than 90 g, or greater than 100 g. Advantageously, the mass of product is at least 40 g.

### Step (g)

Step (g) comprises passing the 5,15-bis-[4-(3-trimethylammonio-propyloxy)-phenyl]-porphyrin dibromide produced in step (f) through an anion exchanger to produce 5,15-bis-[4-(3-trimethylammonio-propyloxy)-phenyl]-porphyrin dichloride.

Suitable anion exchangers are well known in the art, for example Amberlite® anion exchange resins such as IRA-958 (available from Sigma Aldrich, Poole, UK).

In a preferred embodiment of the invention, step (g) comprises dissolving 5,15-bis-[4-(3-Trimethylammonio-propyloxy)-phenyl]-porphyrin dibromide in acetonitrile, methanol and distilled water.

Preferably, the acetonitrile, methanol and distilled water are present in a volume ratio of 1.5:6:1, respectively.

Advantageously, the solution containing 5,15-bis-[4-(3-trimethyl-ammonio-propyloxy)-phenyl]-porphyrin dibromide is heated prior to passing through an anion exchanger. For example, the solution may be heated to at least 40° C., preferably to 50° C.

The dichloride salt of 5,15-bis-[4-(3-trimethylammonio-propyloxy)-phenyl]-porphyrin may be eluted from the anion exchanger with a suitable solvent, such as methanol. The product may then be dried by evaporation of the solvent, for example by rotary evaporation.

Advantageously, the 5,15-bis-[4-(3-trimethylammonio-propyloxy)-phenyl]-porphyrin dichloride is further purified by recrystallisation.

The percentage yield of the dichloride salt in the reaction described above is preferably greater than 50%, for example greater than 55%, greater than 60%, greater than 65%, greater than 70%, greater than 75%, greater than 80%, greater than 85%, greater than 90% or greater than 95%. Advantageously, the yield is at least 80%.

Likewise, the mass of the dichloride salt produced in the reaction described above is preferably greater than 10 g, for example greater than 20 g, greater than 30 g, greater than 40 g, greater than 50 g, greater than 60 g, greater than 70 g, greater than 80 g, greater than 90 g, or greater than 100 g. Advantageously, the mass of product is at least 70 g.

Thus, the present invention provides a process suitable for the large-scale production (i.e. in the gram to kilogram range) of dihalide salts of 5,15-bis-[4-(3-trimethylammonio-propyloxy)-phenyl]-porphyrin, for example dibromide and dichloride salts thereof.

A significant advantage of the process of the invention compared to known methods, such as those described in WO 2004/035590, is the high product yield. For example, the process of the invention permits the preparation of 5,15-bis-[4-(3-trimethylammonio-propyloxy)-phenyl]-porphyrin dichloride in a cumulative yield for steps (a) to (e) of greater than 20%, for example 25%.

A second aspect of the invention provides a process for the production of 5,15-bis-(4-{3-[(3-dimethylamino-propyl)-dimethyl-ammonio]-propyloxy}-phenyl]-porphyrin dihalide, the process comprising steps (a) to (f) as defined above in relation to the first aspect of the invention, wherein in step (f) the trimethylamine is replaced with N,N,N′,N′-tetramethyl-1,3-propanediamine.

In a preferred embodiment of the second aspect of the invention, the process further comprises step (g) of passing the 5,15-bis-(4-{3-[(3-dimethylamino-propyl)-dimethyl-ammonio]-propyloxy}-phenyl]-porphyrin dibromide produced in step (f) through an anion exchanger (such as Amberlite® IRA-958) to produce 5,15-bis-(4-{3-[(3-dimethylamino-propyl)-dimethyl-ammonio]-propyloxy}-phenyl]-porphyrin dichloride.

The invention is illustrated, but in no way limited, by the following examples.

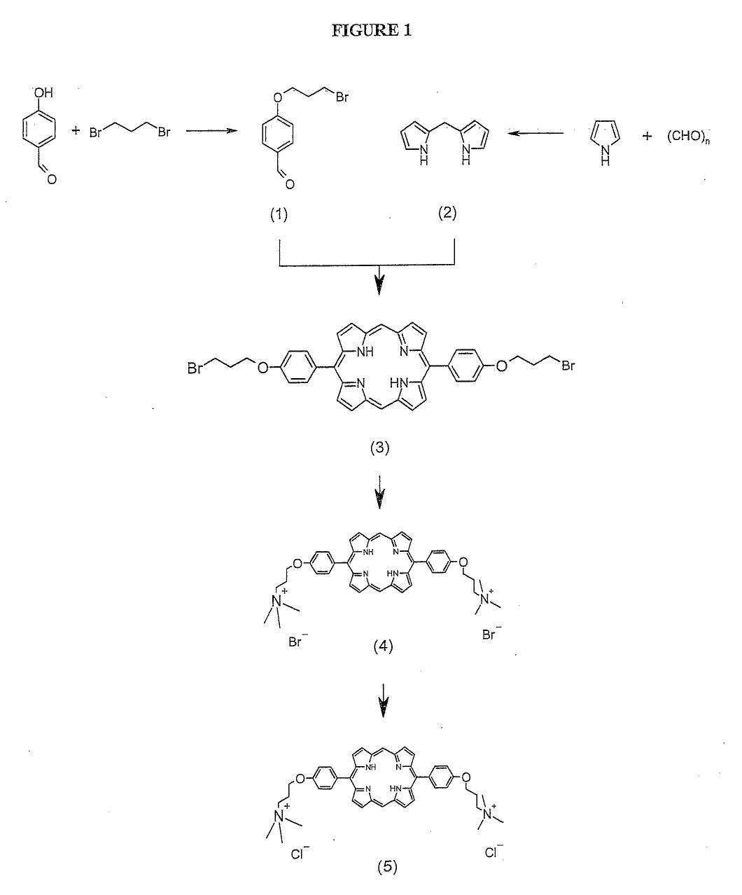

FIG. 1 is a schematic diagram showing the key reaction steps in the synthesis of 5,15-bis-[4-(3-trimethylammonio-propyloxy)-phenyl]-porphyrin dichloride.

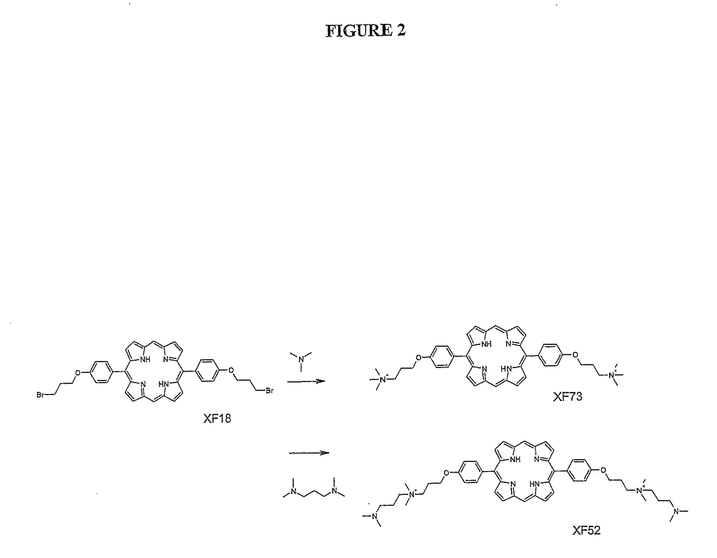

FIG. 2 is a schematic diagram showing an alternative embodiment of the process of the invention for producing 5,15-bis-(4-{3-[(3-dimethylamino-propyl)-dimethylammonio]-propyloxy}-phenyl]-porphyrin dibromide, wherein in step (d) the trimethylamine is replaced with N,N,N′,N′-tetramethyl-1,3-propanediamine.

### EXAMPLE

### Reagents and Chemicals

These were purchased variously from Acros, Merck and Fluka. Solvents were obtained from Schweizerhall.

### Analysis:

Proton NMR spectra were recorded on a Bruker B-ACS60 (300 MHz) instrument using TMS as internal standard. The chemical shifts are given in ppm and coupling constants in Hz in the indicated solvent.

Analytical thin-layer chromatography (TLC) was performed using layers of silica gel (Merck, 60F254). The following solvent systems were employed:

A: Heptane:ethyl acetate (3:1, by vol.) with UV detection at 254 nm

B: Heptane:ethyl acetate-dichloromethane (8:1:1, by vol.) with UV detection at 254 nm

Column chromatography was carried out using silica gel (Merck Silicagel 60, Fluka 60, 0.040-0.063 mm).

Combined Liquid Chromatography/Mass Spectrometry (LC/MS) analyses were performed on an Agilent 110 Series (LC) and Water Micromass ZQ (MS) instrument. Conditions employed were:

(LC) 8 min gradient, 5-100% B. A=H₂O+0.04% HCOOH; B=CH₃CN:CH₃OH (4:1, by vol.)+0.05% HCOOH. Flow rate=1.7 mL/min. Column: YMX-Pack Proc 18, (33×3.0 mm), 3 μm.

Gas chromatography (GC) was performed using a Perkin Elmer AutoSystem XL Gas Chromatograph with a 6×2 mm ID glass column for Autosystem (NOC) which was packed with W—HP 80/100 Mesh 10% OV-101 Silicone; Hydrogen as carrier gas.

Conditions employed were: 60° C. for 1 min, then 16° C./min to 270° C., 270° C. for 8 min.

### ABBREVIATIONS

DDQ=2,3-Dichloro-5,6-dicyano-1,4-benzoquinone

for NMR: (s) singlet, (bs) broad singlet, (d) doublet, (t) triplet, (q) quartet, (quint) quintet, (m) multiplet.

for GC: rt=retention time

for IR: s, strong; ms, medium-strong; m, medium; mw, medium-weak; sh, shoulder; br, broad.

The exemplary process of the invention is shown schematically in FIG. 1.

### Step (a): 4-(3-Bromopropyloxy)benzaldehyde (Compound 1)

A dry Belatech glass reactor (30 L) was flushed with argon and charged with 4-hydroxybenzaldehyde (588 g, 4.8 Mole), 1,3-dibromopropane (4.976 kg, 24.6 Mole) and anhydrous acetonitrile (24 L) under argon. Dried powdered potassium carbonate (1.66 kg, 12 Mole) was added in portions to the stirred solution. The suspension was stirred at 55-60° C. and monitored by gas chromatography and cooled (ice bath) to room temperature as soon as the 4-hydroxybenxaldehyde had been consumed (3-4 hr). Solids were removed by filtration (2 L Buechner funnel) and washed with dry acetonitrile (3×300 mL). The combined solvents were reduced in volume by rotary evaporation (bath temperature 40° C.) and then the excess of 1,3-dibromopropane was removed by high vacuum distillation (bath temperature 40° C.). Crude product was obtained as a bright yellow oil (1350 g). The crude product was purified by column chromatography under argon using 10 kg silica gel, eluting with a mixture of heptane:ethyl acetate (75 L; 9:1, by vol.) followed by a mixture of heptane:ethyl acetate (11 L; 4:1, by vol.). After 5 μL of the first eluant had been eluted, fractions (500 mL) of eluate were collected and their purities checked by TLC. The fractions containing pure product were combined and dried by rotary evaporation (bath temperature 40° C.) to yield pure product as a colourless oil. Yield: 900 g (3.7 Mole, 77%). TLC: Rf=0.38 (A). GC: purity >95% (rt=12.7 min). ¹H-NMR analysis: δ_{H} (300 MHz, CD₃OD): 2.35 (quint, ³J 7.4 Hz, 2H), 3.58 (t, ³J 7.4 Hz, 2H), 4.18 (t, ³J 7.4 Hz, 2H), 6.95, 7.85 (2×d, ³J 8.5 Hz, 4H), 9.85 (s, 1H).

### Comments:

Acetone and THF as reaction solvents were also investigated and found to give inferior outcomes to acetonitrile.

It is important to secure highly pure product in this step. Product contaminated with the elimination product leads in the next step to the corresponding porphyrin by-product containing an unsaturated propene group.

The product is air sensitive. Formation of the oxidation product (the carboxylic acid) was observed during workup. Due to the air sensitivity of the product, column chromatography should be carried out under an argon atmosphere and the bottles of the collected fractions should be kept closed.

Two main by-products, an elimination product and a dimer, formed in the reaction. By TLC analysis, three compounds were observed: 4-allyloxybenzaldehyde (R_{f} 0.42), product (R_{f} 0.38) and 4-[3-(4-formylphenoxy)propyloxy]benzaldehyde (R_{f}=0.20).

By GC analysis four compounds were detected: excess 1,3-dibromopropane (rt=4.525 min); elimination product (rt=9.725 min); product (rt=12.858 min) and the dimer (rt=19.75 min).

### Step (b): Dipyrrolmethane (Compound 2)

A Suko glass reactor (4.5 L) was flushed with argon and charged with pyrrole (3.47 L, 50 Mole) and paraformaldehyde (15 g, 0.5 Mole) at room temperature. Argon was bubbled through the vigorously stirred suspension for 15 mins and it was warmed to 55° C. (bath temperature 61° C.). Indium trichloride (11.1 g, 0.05 Mole) was added in one portion (slightly exothermic) and the reaction mixture was stirred at 50-55° C. for 3 hr. The reaction was monitored by GC (B) and when complete the mixture was cooled (ice bath) to room temperature. Powdered sodium hydroxide (60 g, 1.5 Mole) was added in one portion and the reaction mixture was stirred for another 1.5% at room temperature. The mixture was filtered through a pad of Hyflo Super Cell (Fluka 56678) to remove insoluble matter which was washed with pyrrole (1 L). The filtrate was dried with rotary evaporation (bath temperature 40° C., 50 mbar) to remove the excess of pyrrole and then under high vacuum to complete dryness. A dark brown oil (100 g) was obtained which was dissolved in a mixture of ethyl acetate (40 mL) and heptane (40 mL) and purified by column chromatography using silica gel (1.5 kg) which was eluted with heptane:ethyl acetate (approx. 3.5-4.0 L; 7:1, by vol.) followed by heptane:ethyl acetate (approx. 3.0-4.0 L; 5:1, by vol.). The eluate was collected in fractions (250 mL) and their purities were analysed by TLC. The fractions containing pure product were combined and dried by rotary evaporation to afford product as a light-yellowish solid. Yield: 59.9 g (0.41 Mole, 82%). TLC: Rf=0.25 (B). GC: purity >95% (rt=10.07 min). ¹H-NMR analysis: δ_{H} (300 MHz, CD₃OD): 3.85 (s, 2H), 6.02 (m, 2H), 6.15 (m, 2H), 6.55 (m, 2H), 7.40-7.80 (br, 2H).

### Comments:

The product after chromatography can be used in the next step without further purification.

Indium-catalysed dipyrrolmethane synthesis was found superior to the reaction catalysed by trifluoroacetic acid. Improved yields were obtained and control of the reaction conditions was found to be easier to effect.

The Indium content of the product was analyzed by elemental analysis and no trace (<1 ppm) was found.

The yield was dependent on the source of the indium trichloride. In this study, material from Fluka gave slightly lower yields (˜70%) than that from Merck.

Recovered pyrrole can be re-used.

Purification of the product can be carried out either by column chromatography over silica gel as described or by solid distillation. Using the latter technique, significant decomposition of product was observed and the yields were approx. 10% lower than with column chromatography.

When the reaction was up-scaled to 10 L of pyrrole in a 10 L glass reactor, lower yields (between 60-66%) were obtained.

Further purification can be effected by recrystallisation as the following example: Purified product (14 g) was dissolved in ethanol:water (70 mL; 1:1, by vol.) at 70° C. to give a clear yellow solution. The solution was cooled to room temperature and a few seed crystals were added. The solution was cooled to 0° C. slowly, when a large amount of colourless crystals formed. The suspension was maintained at 0° C. for 1 hr and the crystals were collected by filtration and washed with ethanol:water (1:1; by vol. at 0° C.) and dried under vacuum (100 mbar, 40° C.) overnight to afford the pure product as colourless crystals in a recovery of 79% (11 g).

Steps (c) to (e): 5,15-bis-[4-(3-Bromo-propyloxy)-phenyl]-porphyrin (Compound 3, or C-3)

A glass reactor (10 L) was flushed with argon and charged with dry dichloromethane (7.7 L) at room temperature. Dry argon was passed through the solvent for the remainder of the reaction under vigorous stirring. Compound 1 (9.93 g, 0.067 Mole) and compound 2 (16.7 g, 0.068 Mole) were added and the reaction mixture was stirred for a further 20 min. Trifluoroacetic acid (1.55 mL, 0.020 Mole) was added dropwise. After stirring at room temperature for 15 min, the reaction mixture became dark (within 15-20 min). It was stirred in the dark at room temperature overnight. DDQ (42.8 g, 0.19 Mole) was added in portions. The reaction mixture became black at once and was stirred at room temperature for a further 1 hr at 20° C. The reaction mixture was neutralized with triethylamine (2.46 mL) and stirred for 20 min. Neutral alumina (657 g) was added and the mixture stirred for a further 20 min at 20° C. The reaction mixture was completely dried by rotary evaporation (10 L apparatus) at no more than 40° C. The residue, obtained as a black powder, was continually extracted in two separate portions (Soxhlet) with dichloromethane (2 L) for 5-6 days. After cooling to room temperature, the volume of dichloromethane was reduced by rotary evaporation at 40° C. to 100 mL. After storage at 20° C. for at least 15 min, the crystalline product was collected by filtration using a Buechner funnel. The crystals were washed with acetone (3×10 mL) and then dichloromethane acetone (3×10 mL) until the washings were colourless. Drying under vacuum afforded the product as violet crystals. Yield: 10.68 g (39%, 14.5 mMole). LC/MS analysis: rt=5.77 min, [M+H]+=737; [M+H+CH3CN]+=410. 1H-NMR analysis: □H (300 Mz, d6-DMF): 2.75 (quint, 3J 7.5 Hz, 4H), 4.15 (t, 3J 7.5 Hz, 4H), 4.70 (t, 3J 7.5 Hz, 4H), 7.75, 8.50 (2×d, 3J 8.4 Hz, 2×4H), 9.35, 9.90, (2×d, 3J 7.2 Hz 2×4H), 10.85 (s, 2H).

### Comments:

It is essential to perform the reaction in the absence of oxygen and in the dark (e.g. the reactor is wrapped in aluminium foil). Dry argon or nitrogen is bubbled through the reaction solution during the entire operation. The cyclisation reaction is conducted at optimal concentration as found by investigation. The oxidation by DDQ is conducted at 20° C. for no more than 1 hour at which time triethylamine is added. Aluminium oxide is added to the stirred solution at no later than 20 minutes after the addition of the triethylamine. The suspension is dried by rotary evaporation at 40° C. in the absence of light to give a black powder.

The compound complexes metals. The use of metal spatulas and other metal items should be kept to a minimum.

Other oxidation reagents than DDQ were investigated; e.g. air or O₂/Pt.; H₂O₂ the best procedure was with DDQ.

To remove impurities, Soxhlet extraction is more efficient than filtration through alumina and less solvent is used. An amount of alumina relative to the organic material is added to eliminate chlorination side-reactions during the extraction. This is added to the reaction solution from the cyclisation step before the mixture is dried down to give a powder suitable for Soxhlet extraction. The black powder is continually extracted (Soxhlet) with dichloromethane with daily changing of solvent and in-process control until no more material is eluted that satisfied the purity criterion. Samples of each fraction are monitored by HPLC. Selected fractions are combined and the volume of dichloromethane is reduced and the crude product which crystallises is collected by filtration, washed with acetone and then dichloromethane to remove starting materials. The moist product is dried at no more than 40° C. for at least 2 hours to constant weight.

It is desirable that no more than 0.5% 10,20-di-chloro compound is present as contaminant as in the next synthetic step, amination with trimethylamine, the generated 10,20-di-chloro contaminant cannot be removed by re-crystallisation from the target compound. Any 10-mono-chloro compound present is easily removed on re-crystallisation. It is therefore essential to have an IPC to monitor the presence of the di-chloro compound. If it is present (at the start of the extraction, it is quickly eluted due to its high lipophilicity), the early cut fractions are rejected. If any black material is eluted, the solvent flask must be changed immediately.

The product is poorly soluble in all common organic solvents and crystallises very easily. The crystals are very difficult to re-dissolve.

Thin layer chromatography is conducted on layers of Kiesegel 60 F₂₅₄ developed with dichloromethane. The developed plate is examined by UV at 366 nm. The product fluoresces pink/red when the layer is still damp. ° F. ca. 0.85. Due to the low solubility of the compound, it can streak from the origin.

| Test | Criterion |
|---|---|
| Appearance | Purple solid |
| Identity | By HPLC: |
| | Column: Lichrosorb Si-60-5 150 × 4.6 mm ID |
| | Mobile Phase: n-Hexane:Dichlromethane:Tetrahydrofuran: |
| | Trifloroacetic acid (600:200:200:1, by vol.) |
| | Flow rate: 1.0 mL/min |
| | Detection: 420 nm |
| | Injection Volume: 10 μL of 1 mg/mL solution |
| | Acquisition time: 45 minutes |
| | Elution times: C-3 = 21.31 minutes; 10-chloro C-3 = 26.89 |
| | minutes; 10,20 dichloro C-3 = 38.35 minutes |
| Purity | <0.5% 10,20-dichloro C-3 & <20% 10-chloro C-3 |

Other oxidation reagents than DDQ were investigated; e.g. air or O₂/Pt.; H₂O₂: the best procedure was with DDQ.

The cyclisation reaction is at optimal concentration as described in the above synthesis.

Step (f): 5,15-bis-[4-(3-Trimethylammonio-propyloxy)-phenyl]-porphyrin dibromide (Compound 4, or C-4)

Molecular sieve (UOP Type 4A [Fluka 69838], 227 g) was added to dimethylformamide (11.4 L) and the mixture was stirred for 1 hr at room temperature. The suspension was stored overnight. A dried autoclave (20 L) was is flushed with dry nitrogen and charged with the dry dimethylformamide. Compound 3 (37.11 g, 0.0495 Mole) was suspended in the solvent and trimethylamine (515 g, 8.71 Mole) was added slowly from a steel cylinder via a steel pipe. The reaction mixture was stirred at 50° C. for 17 hr (pressure=1-2 bar). The reaction was monitored by LC/MS. After cooling the reaction mixture to room temperature, the excess of trimethylamine was removed by rotary evaporation at no more than 50° C. under reduced pressure (10-15 mbar). The crude product suspended as violet crystals in the reaction mixture was collected by filtration using a Buechner funnel. The crude product was washed with dichloromethane (3×360 mL) and then dried to constant weight at 40° C. in vacuo to give 42.83 g (0.051 Mole, 101%) of violet crystals. LC/MS analysis: rt=2.14 min, [M]<=347.4, [M]⁺⁺⁺=232. ¹H-NMR analysis: δ_{H} (300 Mz, CD₃OD): 2.40-2.60 (m, 4H), 3.30-3.35 (bs, 18H), 3.75-3.80 (m, 4H), 4.40 (t, 3J 7.5 Hz, 4H), 7.40, 8.20 (2×d, 3J 8.5 Hz, 8H), 9.05, 9.50 (2×d, 3J 4.5 Hz, 8H), 10.45 (s, 2H).

### Comments:

Dry DMF is essential for the reaction to ensure the precipitation of almost all of product and to avoid corrosion of metal autoclave which gives rise to metal complexes of the final product as impurities. The use of metal spatulas and other metal items must be avoided.

The construction material of the autoclave should be carefully considered. The product is an excellent co-ordinator for many metal ions. Use of an all-glass autoclave is preferred. Vessels constructed of Hastelloy C or E are also suitable. The pressure in the autoclave is dependent on the size of autoclave used. Excess pressure is not necessary for reaction.

The product has very low solubility in DMF at room temperature. Provided the DMF used is sufficiently dry, the product can be collected by filtration directly from the reaction mixture (normally over 90-95% of the product is precipitated).

| Test | Criterion | | | |
|---|---|---|---|---|
| Appearance | Purple solid | | | |
| Identity | By HPLC: | | | |
| | Column: Symmetry C8, 250 × 4.6 mm ID | | | |
| | Mobile Phase A: Water:Tetrahydrofuran (85:15, by vol.) | | | |
| | +0.1% Trifloroacetic acid +1 g/L Hexanesulfonic | | | |
| | acid sodium salt monohydrate | | | |
| | Mobile Phase A: Acetonitrile:Tetrahydrofuran:Water | | | |
| | (65:15:20, by vol.) +0.1% Trifloroacetic acid +1 g/L | | | |
| | Hexanesulfonic acid sodium salt monohydrate | | | |
| | Gradient Profile: | Time (min) | % A | % B |
| | | 0 | 95 | 5 |
| | | 15 | 55 | 45 |
| | | 35 | 10 | 90 |
| | | 36 | 95 | 5 |
| | | 41 | 95 | 5 |

| | |
|---|---|
| | Flow rate: 1.0 mL/min |
| | Detection: 420 nm |
| | Injection Volume: 10 μL of 1 mg/mL solution |
| | Acquisition time: 35 minutes |
| | Elution times: C-4 = 6.92 minutes; 10-chloro C-4 = 7.93 |
| | minutes |
| | By 1H-NMR: |
| | In CH₃OD or DMSO-d₆ |
| Purity | Material is purified as the dichloride salt C-5. Hence, |
| | there is no specification for the intermediate di-bromide |
| | salt C-4 |

Step (g): 5,15-bis-[4-(3-Trimethylammonio-propyloxy)-phenyl]-porphyrin dichloride (Compound 5, or C-5)

Compound 4 (42.83 g, 48.7 mMole) was dissolved in a mixture of acetonitrile:methanol:doubly-distilled water (1.5:6:1 by vol., 2005 mL) and the solution was passed through a column (height 27 cm, diameter 10 cm) of anion exchanger (1.4 kg, IRA-958 chloride form) eluting with methanol (9 L). The resulting solution was evaporated to complete dryness by rotary evaporation (bath temperature 40-50° C.). The crude product was obtained as a violet solid (Yield 35.68 g (46.6 mMol, 95.7%)).

Raw product (35.68 g) was re-dissolved in a mixture of acetonitrile:methanol:doubly-distilled water (1.5:1.5:0.05, by vol., 970 mL) and the solution was stirred at 50° C. for 15 min. Toluene (1.355 L) was slowly added at 50° C. during 45 min. The volume of the solution was then slowly reduced under vacuum (400 mbar, 50° C., rate 250 mL/hr) to remove 63-68% of the volume of the added toluene. The mixture was cooled to 20° C. Crystalline material was collected by filtration. After drying, the purity was assessed by HPLC. Recrystallisation was repeated using the same conditions but lowering the amount of toluene removed by distillation until material met the specification for content and level of defined impurities. The product failed to meet the specification for toluene content even after drying for a prolonged period under high vacuum. It was finally re-crystallised using the original condition (removal of 68% of the volume of the added toluene) and then dried under high vacuum (40° C., 0.1 mbar, 2 hr). The product was obtained as violet crystals (24.23 g) in a recovery of 67.9%. ¹H-NMR analysis: δ_{H} (300 Mz, CD₃OD): 2.40-2.60 (m, 4H), 3.30-3.35 (bs, 18H), 3.75-3.80 (m, 4H), 4.40 (t, 3J 7.5 Hz, 4H), 7.40, 8.20 (2×d, 3J 8.5 Hz, 8H), 9.05, 9.50 (2×d, 3J 4.5 Hz, 8H), 10.45 (s, 2H). ¹³C-NMR analysis: 6 (75 Mz, CD₃OD): 24.52, 53.74, 65.67, 66.03, 106.41, 114.37, 119.96, 131.86, 133.05, 135.41, 137.06, 146.49, 160.07. IR analysis: (cm⁻¹): 3600-3300 (br, s), 3150-2800 (w), 1604 (s), 1600-1500 (m, sh), 1480-1410 (s, ms, m), 1230-1220 (s, sh), 1176, 1145, 1110 (ms, m, s), 1054, 972, 956, 918 (ms, m, s), 731 (ms), 720 (mw, w). ESI-MS analysis: M⁺⁺/Z=347.5, [M+H]⁺⁺⁺/Z=232. Melting point: 127.2° C.

### Comments:

The compound complexes metals. Metal spatulas should not be used and the compound should be handled in Hastelloy C or plastic vessels. The Amberlite IRA 958 chloride form ion exchange resin is washed sequentially before use with ethanol:acetonitrile:methanol:water (1.5:6:1, by volume) and methanol. Compound C-5 is applied to the column dissolved in acetonitrile:methanol:water (1.5:6:1, by volume) and the bed is eluted with methanol until the eluate is colourless. The eluate is evaporated below 50° C., the residue is dissolved in a mixture of acetonitrile:methanol:water (1.5:1.5:0.05, by volume) at 50° C. with stirring and after 15 minutes, toluene is added at 50° C. slowly over 45 minutes. The mixture is distilled at 57° C. at a maximum of 400 mbar and 63-68% of the volume of toluene is distilled off as required. The residual solution is cooled to 20° C. and solid material collected by filtration and the filter cake dried in a stream of nitrogen. Purity is assessed by HPLC analysis at 420 nm. This provides an overestimate of impurities, especially those containing chlorine at the bridgehead positions. The material is re-crystallised by dissolving the material in 23 mL/g of acetonitrile:methanol:water (1.5:1.5:0.05, by volume) and then adding 33 mL/g of toluene and distilling off 31-68% of the volume of toluene as required until the product satisfies the criteria of purity for related impurities.

Re-crystallisation of the material to obtain product within specification from the point of view of by-products proceeds well but, the material obtained does not meet the specification for residual solvents (toluene only). A final crystallisation under original (63-68% removal of the volume of added toluene) conditions permits isolation of material within specification in all respects, i.e. C-5 content is greater than 99.0%, 10-chloro C-5 content is less than 0.30% and no other single contaminant is present at greater than 0.3% assessed by HPLC analysis with UV detection at 420 nm.

Due to the high affinity of compounds C-3, C-4 and C-5 for metals, it is recommended that the HPLC systems used for their analysis have undergone a passivation procedure using 6M nitric acid within the previous 12 months.

1. A process for the preparation of 5,15-bis-[4-(3-trimethylammonio-propyloxy)-phenyl]-porphyrin dihalide, wherein the process comprises the following steps:
(a) providing 4-(3-bromopropyloxy)benzaldehyde;
(b) providing dipyrrolmethane;
(c) reacting the 4-(3-bromopropyloxy)benzaldehyde with the dipyrrolmethane, together with trifluoroacetic acid;
(d) adding an oxidation reagent to produce 5,15-bis-[4-(3-bromo-propyloxy)-phenyl]-porphyrin;
(e) purifying the 5,15-bis-[4-(3-bromo-propyloxy)-phenyl]-porphyrin produced in step (d) by Soxhlet extraction in the presence of aluminium oxide; and
(f) reacting the purified 5,15-bis-[4-(3-bromo-propyloxy)-phenyl]-porphyrin with trimethylamine in the presence of dry dimethylformamide to produce 5,15-bis-[4-(3-trimethylammonio-propyloxy)-phenyl]-porphyrin dibromide
wherein step (e) comprises monitoring of Soxhlet extracted fractions to determine the presence therein of contaminants.

2. A process according to claim 1 further comprising step (g) of passing the 5,15-bis-[4-(3-trimethylammonio-propyloxy)-phenyl]-porphyrin dibromide produced in step (d) through an anion exchanger to produce 5,15-bis-[4-(3-trimethylammonio-propyloxy)-phenyl]-porphyrin dichloride.

3. A process according to any one of the preceding claims wherein in step (a) the 4-(3-bromopropyloxy)benzaldehyde is at least 95% pure.

4. A process according to any one of the preceding claims wherein in step (a) the 4-(3-bromopropyloxy)benzaldehyde is prepared by reaction of 4-hydroxybenzaldehyde and 1,3-dibromopropane in an inert atmosphere.

5. A process according to claim 4 wherein the 4-hydroxybenzaldehyde and 1,3-dibromopropane are reacted in a molar ratio of between 1:4 to 1:6, preferably in a molar ratio of 1:5.

6. A process according to claim 4 or 5 wherein the reaction is performed under argon.

7. A process according to any one of claims 4 to 6 wherein the reaction is performed in anhydrous acetonitrile.

8. A process according to any one of claims 4 to 7 wherein the reaction is performed at a temperature of between 55 and 60° C.

9. A process according to claim 8 wherein the reaction is performed for between 3 to 4 hours.

10. A process according to any one of claims 4 to 9 wherein the reaction is monitored by gas chromatography.

11. A process according to any one of claims 4 to 10 wherein the reaction is cooled to room temperature upon completion.

12. A process according to any one of claims 4 to 11 wherein the 4-(3-bromopropyloxy)benzaldehyde is purified from the reaction mixture by removal of solids by filtration, reduction of the solvent volume by rotary evaporation and removal of excess 1,3-dibromopropane by high vacuum distillation.

13. A process according to claim 12 wherein the 4-(3-bromopropyloxy)benzaldehyde is further purified by column chromatography under argon and pooling of elution fractions containing pure product.

14. A process according to any one of claims 4 to 13 wherein the yield of 4-(3-bromopropyloxy)benzaldehyde is greater than 70%, for example at least 75%.

15. A process according to any one of claims 4 to 14 wherein the yield of 4-(3-bromopropyloxy)benzaldehyde is greater than 500 g, for example at least 900 g.

16. A process according to any one of the preceding claims wherein in step (b) the dipyrrolmethane is at least 85% pure.

17. A process according to any one of the preceding claims wherein in step (b) the dipyrrolmethane is prepared by reaction of pyrrole with paraformaldehyde in an inert atmosphere.

18. A process according to claim 17 wherein the pyrrole and paraformaldehyde are reacted in a molar ratio of between 120:1 to 80:1, preferably in a molar ratio of 100:1.

19. A process according to claim 17 or 18 wherein the reaction is performed under argon.

20. A process according to any one of claims 17 to 19 wherein the reaction is catalysed using an indium-based catalyst

21. A process according to claim 20 wherein the catalyst is indium trichloride.

22. A process according to any one of claims 17 to 21 wherein the reaction is performed at a temperature of between 50 and 55° C.

23. A process according to any one of claims 17 to 22 wherein the reaction is monitored by gas chromatography.

24. A process according to any one of claims 17 to 23 wherein the reaction is cooled to room temperature upon completion.

25. A process according to claim 24 wherein sodium hydroxide is added after cooling of the reaction mixture.

26. A process according to any one of claims 17 to 25 wherein the dipyrrolmethane is purified from the reaction mixture by removal of solids by filtration, removal of excess pyrrole from the filtrate by rotary evaporation and then drying under high vacuum.

27. A process according to claim 26 wherein the dipyrrolmethane is further purified by column chromatography and pooling of elution fractions containing pure product.

28. A process according to claim 26 wherein the dipyrrolmethane is further purified by solid distillation.

29. A process according to any one of claims 26 to 28 wherein the dipyrrolmethane is further purified by recrystallisation.

30. A process according to any one of claims 17 to 29 wherein the yield of dipyrrolmethane is greater than 60%.

31. A process according to claim 30 wherein the yield of dipyrrolmethane is greater than 80%.

32. A process according to any one of claims 17 to 31 wherein the yield of dipyrrolmethane is greater than 50 g.

33. A process according to claim 32 wherein the yield of dipyrrolmethane is greater than 60 g.

34. A process according to any one of the preceding claims wherein in steps (c) to (e) are performed in the dark and in the absence of oxygen.

35. A process according to any one of the preceding claims wherein in steps (c) to (e) the reaction is performed under argon.

36. A process according to any one of the preceding claims wherein in steps (c) to (e) the reaction is performed in dichloromethane.

37. A process according to any one of the preceding claims wherein in step (c) the 4-(3-bromopropyloxy)benzaldehyde and dipyrrolmethane are reacted in a molar ratio of 1:1.

38. A process according to any one of the preceding claims wherein in step (c) the 4-(3-bromopropyloxy)benzaldehyde and dipyrrolmethane are reacted at a concentration of between 7 and 10 mmol/L.

39. A process according to claim 38 wherein in step (c) the 4-(3-bromopropyloxy)benzaldehyde and dipyrrolmethane are reacted at a concentration of 8.75 mmol/L.

40. A process according to any one of the preceding claims wherein in step (d) the oxidation reagent is added after the reaction mixture has been stirred at room temperature for at least 16 hours.

41. A process according to any one of the preceding claims wherein in step (d) the oxidation reagent is 2,3-dichloro-5,6-dicyano-1,4-benzoquinone.

42. A process according to any one of the preceding claims wherein in step (d) the reaction is neutralised within one hour of addition of the oxidation reagent.

43. A process according to any one of the preceding claims wherein in step (d) the reaction mixture is neutralised by the addition of triethylamine following addition of the oxidation reagent.

44. A process according to any one of the preceding claims wherein in step (c) aluminium oxide is added to the reaction mixture after completion of the reaction.

45. A process according to claim 44 wherein the aluminium oxide is added within 20 minutes of neutralisation of the oxidation reaction.

46. A process according to any one of the preceding claims wherein in step (d) the reaction mixture is dried after completion of the reaction by rotary evaporation.

47. A process according to claim 46 wherein the rotary evaporation is performed at a temperature not exceeding about 40° C.

48. A process according to any one of the preceding claims wherein in step (e) the Soxhlet extraction is performed with dichloromethane.

49. A process according to any one of the preceding claims wherein in step (e) the Soxhlet extraction is performed for at least 5 days.

50. A process according to any one of the preceding claims wherein in step (e) the monitoring for contaminants is performed by HPLC.

51. A process according to any one of the preceding claims wherein in step (e) the monitoring for contaminants comprises assaying for the presence of the 10,20-dichloro analogue of 5,15-bis-[4-(3-bromo-propyloxy)-phenyl]-porphyrin.

52. A process according to claim 51 wherein Soxhlet extracted fractions comprising more than 0.5% of the 10,20-dichloro analogue of 5,15-bis-[4-(3-bromo-propyloxy)-phenyl]-porphyrin are discarded prior to step (f).

53. A process according to any one of the preceding claims wherein, after Soxhlet extraction, the volume of dichloromethane is reduced by rotary evaporation and the 5,15-bis-[4-(3-bromo-propyloxy)-phenyl]-porphyrin then crystallised and collected by filtration.

54. A process according to claim 53 wherein the rotary evaporation is performed at a temperature not exceeding about 40° C.

55. A process according to any one of the preceding claims wherein the yield in step (c) is greater than 40%, for example at least 45%.

56. A process according to any one of the preceding claims wherein the yield in step (c) is greater than 30 g, for example at least 35 g.

57. A process according to any one of the preceding claims wherein step (f) is performed under argon.

58. A process according to any one of the preceding claims wherein in step (f) the dimethylformamide has been pre-treated with a molecular sieve.

59. A process according to any one of the preceding claims wherein in step (f) the 5,15-bis-[4-(3-bromo-propyloxy)-phenyl]-porphyrin and trimethylamine are reacted in a molar ratio of 1:150 to 1:250, for example in a molar ratio of 1:200.

60. A process according to claim 59 wherein the 5,15-bis-[x-(3-bromo-propyloxy)-phenyl]-porphyrin is reacted at a concentration of between 3 mmol/L and 5 mmol/L.

61. A process according to claim 60 wherein the 5,15-bis-[x-(3-bromo-propyloxy)-phenyl]-porphyrin is reacted at a concentration of 4 mmol/L.

62. A process according to any one of the preceding claims wherein step (f) is performed at a temperature of 50° C. and a pressure of 1 to 2 bar.

63. A process according to any one of the preceding claims wherein in step (f), the reaction is allowed to proceed for at least 10 hours.

64. A process according to claim 63 wherein in step (f) the reaction is allowed to proceed for at least 20 hours.

65. A process according to any one of the preceding claims wherein step (f) is performed in an autoclave.

66. A process according to claim 63 wherein the chamber of the autoclave is made of glass.

67. A process according to any one of the preceding claims wherein in step (f), the excess trimethylamine is removed under vacuum following completion of the reaction.

68. A process according to any one of the preceding claims wherein in step (f), the 5,15-bis-[4-(3-trimethylammonio-propyloxy)-phenyl]-porphyrin dibromide is purified by filtration.

69. A process according to any one of the preceding claims wherein the yield in step (f) is greater than 90%.

70. A process according to claim 69 wherein the yield in step (i) is at least 95%.

71. A process according to any one of the preceding claims wherein the yield in step (f) is greater than 30 g.

72. A process according to claim 71 wherein the yield in step (f) is at least 40 g.

73. A process according to any one of claims 2 to 72 wherein in step (g) the anion exchanger is an Amberlite® anion exchange resin.

74. A process according to claim 73 wherein the anion exchanger is IRA-958.

75. A process according to any one of claims 2 to 74 wherein in step (g) the 5,15-bis-[4-(3-trimethylammonio-propyloxy)-phenyl]-porphyrin dibromide is dissolved in acetonitrile, methanol and distilled water.

76. A process according to claim 74 wherein the acetonitrile, methanol and distilled water are present in a volume ratio of 1.3:7.6:1, respectively

77. A process according to any one of claims 2 to 76 wherein the 5,15-bis-[4-(3-trimethylammonio-propyloxy)-phenyl]-porphyrin dibromide is heated to 50° C. prior to passing through the anion exchanger.

78. A process according to any one of claims 2 to 77 wherein the product is eluted from the anion exchanger with methanol.

79. A process according to any one of claims 72 to 78 wherein the 5,15-bis-[4-(3-trimethylammonio-propyloxy)-phenyl]-porphyrin dichloride is isolated from solution by rotary evaporation.

80. A process according to claim 79 wherein the 5,15-bis-[4-(3-trimethylammonio-propyloxy)-phenyl]-porphyrin dichloride is further purified by recrystallisation.

81. A process according to any one of claims 2 to 80 wherein the yield in step (g) is greater than 70%.

82. A process according to claim 81 wherein the yield in step (g) is at least 80%.

83. A process according to any one of claims 2 to 81 wherein the yield in step (g) is greater than 50 g.

84. A process according to claim 83 wherein the yield in step (g) is at least 70 g.

85. A process according to any one of the preceding claims wherein the overall yield is greater than 20%.

86. A process according to claim 85 wherein the overall yield is greater than 25%.

87. A process for the production of 5,15-bis-(4-{3-[(3-dimethylamino-propyl)-dimethyl-ammonio]-propyloxy}-phenyl]-porphyrin dihalide, comprising a process according to any one of claims 1 to 86 wherein in step (f) the trimethylamine is replaced with N,N,N′,N′-tetramethyl-1,3-propanediamine.

88. A process according to claim 87 further comprising step (g) of passing the 5,15-bis-(4-{3-[(3-dimethylamino-propyl)-dimethyl-ammonio]-propyloxy}-phenyl]-porphyrin dibromide produced in step (i) through an anion exchanger to produce 5,15-bis-(4-{3-[(3-dimethylamino-propyl)-dimethyl-ammonio]-propyloxy}-phenyl]-porphyrin dichloride.

89. A process substantially as described herein with reference to the Example.
